(19) United States
(12) Patent Application Publication
(10) Pub. No.: US 2025/0213824 A1
(43) Pub. Date: Jul. 3, 2025

(54) MEDICAL DRAINAGE TUBE ATTACHMENT DEVICE AND RELATED SYSTEMS AND METHODS

(71) Applicant: MD Biosciences Innovalora, Ltd., Rehovot (IL)

(72) Inventors: David Castel, Tel-Aviv (IL); Sigal Meilin, Givataim (IL)

(21) Appl. No.: 19/003,032

(22) Filed: Dec. 27, 2024

Related U.S. Application Data
(60) Provisional application No. 63/615,340, filed on Dec. 28, 2023.

(51) Int. Cl. A61M 25/02

(52) U.S. Cl. CPC A61M 25/02

(57) ABSTRACT

Various tube attachment device embodiments for attaching a tube, such as a medical drainage tube, to a patient to help stabilize or otherwise maintain the position of the tube in relation to the patient. One device embodiment includes a tube attachment ring comprising a lumen, an extendable clamp arm operably coupled to the tube attachment ring, and a base operably coupled to the tube attachment ring. Another implementation includes a device body comprising a first channel defined within the device body and a clamp body rotatably coupled to the device body, the clamp body comprising a second channel parallel with the first channel. Other aspects include methods of using any such device embodiment to attach a tube to a patient.

### CROSS-REFERENCE TO RELATED APPLICATION(S)

This application claims the benefit under 35 U.S.C. § 119 (e) to U.S. Provisional Application 63/615,340, filed Dec. 28, 2023 and entitled “Chest Tube Attachment Device and Related Systems and Methods,” which is hereby incorporated herein by reference in its entirety.

### FIELD

The various embodiments herein relate to medical devices, and more specifically to attachment devices for ensuring that any externally connected tube or tubular device inserted into a patient is stably attached to the patient so that the attachment device reduces or prevents inadvertent removal of the tube or tubular device.

### BACKGROUND

Pneumothorax is a collection of air outside a patient's lung but within the pleural cavity, while hemothorax is the collection of blood in the pleural cavity. This accumulation of fluid can apply pressure to the lung, potentially leading to collapse.

Treatment of pneumothorax and/or hemothorax primarily involves the insertion of a chest tube (or intercostal drain) in an area under the axilla (armpit) called the “safe triangle”, where damage to internal organs can be avoided. Typically, in the case of spontaneous pneumothorax, small-bore tubes (smaller than 14 F, 4.7 mm diameter) are used, while larger tubes (28 F, 9.3 mm) are generally used for traumatic pneumothorax.

Once the tube is inserted into the patient's chest as desired, it is important to ensure that the tube is attached to the patient such that the tube cannot be inadvertently pulled out of or otherwise retracted from the patient's chest. In case of injury resulting in pneumothorax that occurs outside a sterile hospital environment (such as on a battle field or in an accident, the patient's skin can be dirty, sweaty, covered in blood, or otherwise in a condition that makes attachment of the chest tube difficult. As a result, there are three different chest tube attachment approaches: suturing the tube to the patient's skin, fixing the tube locally to the skin using Vaseline and cotton dressing, or tying the tube to the patient's body using a cotton bandage.

Other similar conditions can also be treated with the use of a drainage tube, including, for example, pleural effusion, infectious effusion, heart failure-related effusion, chylothorax, post-operative thoracic fluid accumulation, bronchopleural fistula, hydropneumothorax, post-thoracentesis complications, lung abscess rupture, and mediastinal fluid accumulation, or any other fluid accumulation in any body part of a patient that can be drained with a tube.

There is a need in the art for improved devices, systems, and methods of attaching a drainage tube to a patient.

### BRIEF SUMMARY

Discussed herein are various tube attachment devices that can be used to attach a tube—such as a medical tube—to a patient such that the tube is stabilized or maintained in position in relation to the patient, along with methods of using such devices to attach such tubes to the patient.

In Example 1, a tube attachment device comprises a tube attachment ring comprising a deformable wall, a lumen defined through the deformable wall, a side opening defined in the deformable wall, wherein the side opening is in fluidic communication with the lumen, and a wall attachment structure disposed on an outer surface of the deformable wall. The tube attachment device further comprises an extendable clamp arm operably coupled to the tube attachment ring, the extendable clamp arm comprising an arm attachment structure disposed on the extendable clamp arm, wherein the arm attachment structure is coupleable with the wall attachment structure, wherein the extendable clamp arm is moveable between a retracted position and an extended position in which the arm attachment structure is coupled to the wall attachment structure and the extendable clamp arm is disposed across the side opening and a base operably coupled to the tube attachment ring, the base comprising a device body attachment component on an underside of the base.

Example 2 relates to the tube attachment device according to Example 1, wherein the wall attachment structure comprises a plurality of teeth.

Example 3 relates to the tube attachment device according to Example 2, wherein the arm attachment structure comprises a projection, wherein the projection is coupleable with the plurality of teeth.

Example 4 relates to the tube attachment device according to Example 1, wherein the extendable clamp arm comprises a handle disposed on the extendable clamp arm.

Example 5 relates to the tube attachment device according to Example 1, wherein the extendable clamp arm in the retracted position is in an untensioned state, and wherein the extendable clamp arm in the extended position is in a tensioned state.

Example 6 relates to the tube attachment device according to Example 1, wherein the device body attachment component is configured to be attachable to a patient's skin.

In Example 7, a tube attachment device comprises device body comprising a first channel defined within the device body, a device base comprising a device body attachment component on an underside of the device base, and a first attachment structure disposed on the device body. Further, the device also comprises a clamp body rotatably coupled to the device body via a rotatable joint, wherein the clamp body is rotatable between an open position and a clamped position, the clamp body comprising a second channel defined within the clamp body, wherein the second notch is parallel with the first channel, and a prong extending from the clamp body, wherein the prong comprises a second attachment structure, wherein the second attachment structure is removably coupleable with the first attachment structure. In addition, when the clamp body is in the clamped position, the first and second channels form a tube lumen sized and shaped to receive a target medical tube such that the device body and the clamp body frictionally attach the tube attachment device to the target medical tube.

Example 8 relates to the tube attachment device according to Example 7, wherein the device body further comprises a tube slot defined within the device base, wherein the tube slot is in communication with the first channel, wherein the tube slot has a length that is transverse to the longitudinal axis of the first channel.

Example 9 relates to the tube attachment device according to Example 7, wherein the device body further comprises an outer wall disposed on the device base, wherein the first attachment structure is disposed on the outer wall.

Example 10 relates to the tube attachment device according to Example 7, wherein the first attachment structure and the second attachment structure comprise mateable teeth.

Example 11 relates to the tube attachment device according to Example 7, wherein the prong is curved to substantially match a curvature of an outer wall of the device body.

Example 12 relates to the tube attachment device according to Example 7, wherein the device body further comprises at least one prong receiving slot associated with the device body, wherein the at least one prong receiving slot is configured to receive the prong as the clamp body is moved into the clamped position.

Example 13 relates to the tube attachment device according to Example 7, wherein the device body attachment component is configured to be attachable to a patient's skin.

Example 14 relates to the tube attachment device according to Example 7, wherein the device body attachment component comprises at least one of an adhesive, microneedles, and microhooks.

In Example 15, a method of ensuring a medical tube inserted into a patient remains attached to the patient comprises urging a tube attachment device toward the medical tube extending from the patient, wherein the tube attachment device comprises a tube attachment ring comprising a lumen defined within the tube attachment ring, a side opening defined in the side of the tube attachment ring, wherein the side opening is in fluidic communication with the lumen, and a first attachment structure disposed on the tube attachment ring. The tube attachment device further comprises an extendable clamp arm operably coupled to the tube attachment ring, wherein the clamp body is movable between an open position and a clamped position, the clamp arm comprising a second attachment structure disposed on the extendable clamp arm. The method further comprises positioning the tube attachment ring such that the medical tube is disposed within the lumen through the side opening, urging the extendable clamp arm toward the clamped position until the tube attachment ring is in sufficient frictional attachment with the medical tube such that the tube attachment device is attached to the medical tube, attaching the extendable clamp arm to the tube attachment ring in the clamped position by attaching the first attachment structure and the second attachment structure, and attaching the tube attachment device to the patient.

Example 16 relates to the method according to Example 15, wherein the urging the extendable clamp arm toward the clamped position further comprises urging the extendable clamp arm into a position in relation to the tube attachment ring such that the second attachment structure is coupled with the first attachment structure, whereby the extendable clamp arm is removably attached to the tube attachment ring.

Example 17 relates to the method according to Example 15, wherein the tube attachment device further comprises a base operably coupled to the tube attachment ring, wherein the base comprises a tube slot in communication with the lumen, wherein the positioning the tube attachment ring such that the medical tube is disposed within the lumen through the side opening further comprises positioning the base such that the medical tube is urged into the tube slot and toward the side opening and the lumen.

Example 18 relates to the method according to Example 15, wherein the tube attachment device further comprises a base operably coupled to the tube attachment ring, wherein the attaching the tube attachment device to the patient further comprises attaching an underside of the base to skin of the patient via an adhesive.

Example 19 relates to the method according to Example 15, wherein the medical tube comprises a chest tube, a dialysis tube, a catheter line, or a tube for a cranial procedure.

Example 20 relates to the method according to Example 15, further comprising detaching the extendable clamp arm from the tube attachment ring by urging a distal end of the extendable clamp arm radially away from the tube attachment ring, whereby the second attachment structure disengages from the first attachment structure.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1A is a perspective top view of a tube attachment device in a clamped position, according to one embodiment.

FIG. 1B is another perspective top view of the tube attachment device of FIG. 1A in an open position, according to one embodiment.

FIG. 2A is a perspective bottom view of the tube attachment device of FIG. 1A in the clamped position, according to one embodiment.

FIG. 2B is another perspective bottom view of the tube attachment device of FIG. 1A in the open position, according to one embodiment.

FIG. 3 is a bottom view of the tube attachment device of FIG. 1A in the clamped position, according to one embodiment.

FIG. 4A is a perspective side view of another tube attachment device in an open position, according to an alternative embodiment.

FIG. 4B is a perspective top view of the tube attachment device of FIG. 4A in the open position, according to an alternative embodiment.

FIG. 5A is a top view of the tube attachment device of FIG. 4A in the open position, according to an alternative embodiment.

FIG. 5B is a bottom view of the tube attachment device of FIG. 4A in the open position, according to an alternative embodiment.

FIG. 6A is a side view of the tube attachment device of FIG. 4A, according to an alternative embodiment.

FIG. 6B is another side view of the tube attachment device of FIG. 4A from the opposite side in comparison to FIG. 6A, according to an alternative embodiment.

### DETAILED DESCRIPTION

The various implementations herein relate to adjustable tube attachment devices for securely attaching a tube to a patient while also allowing for use of the devices with tubes of various sizes. While certain of the devices and methods disclosed or contemplated herein are used for attachment of chest tubes, those of ordinary skill will understand that the various implementations herein can also be used with other types of tubes, including, but not limited to, drainage tubes and the like, that are inserted into other body parts for treating other conditions.

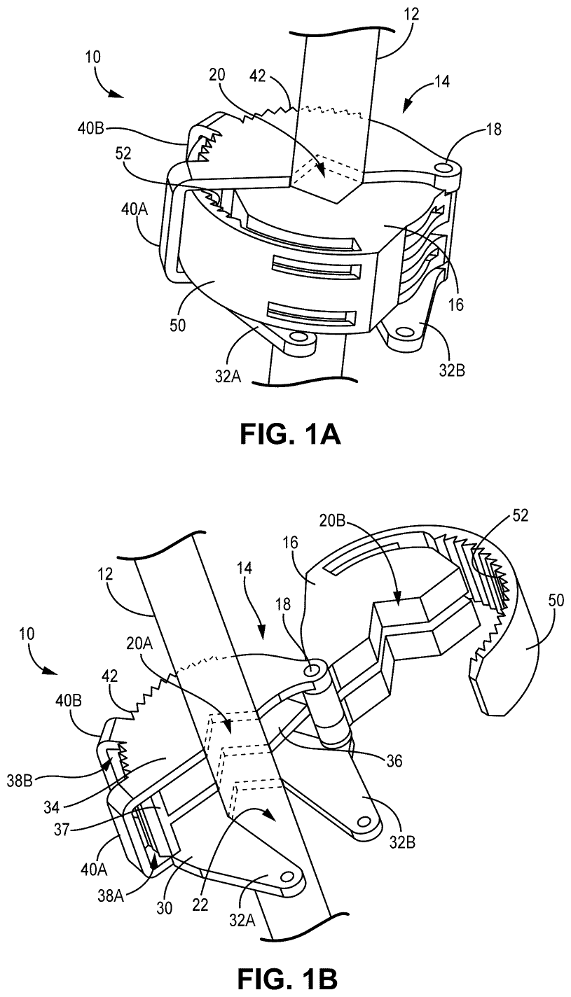

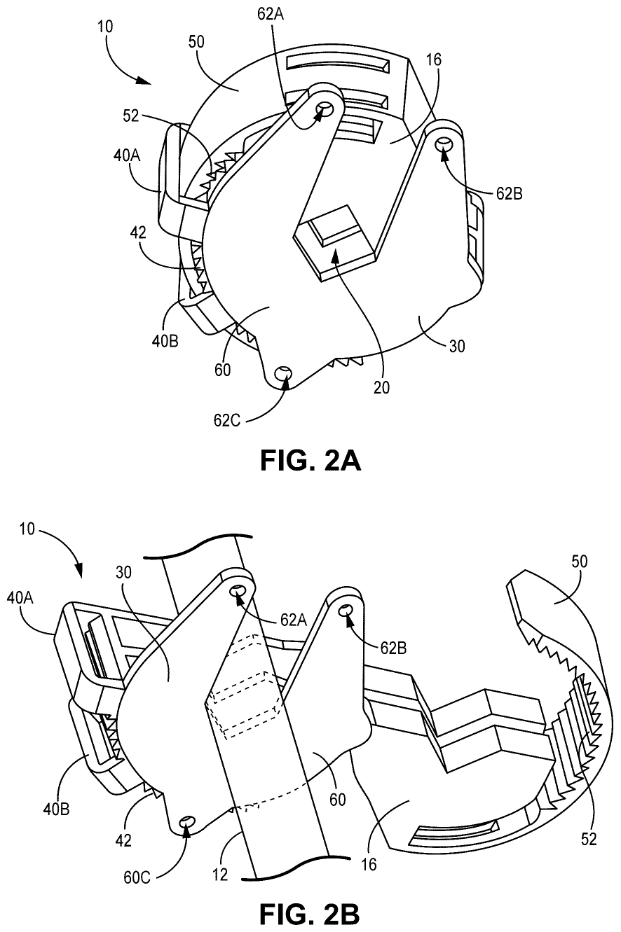

One exemplary embodiment of a tube attachment device 10 is depicted in detail in FIGS. 1A-3. In FIGS. 1A, 2A, and 3, according to one embodiment, the device 10 is shown in its clamped or closed position (with a tube 12 positioned through the device 10), while FIGS. 1B and 2B depict the device 10 in its unclamped or open position, in accordance with another embodiment.

The device 10 in accordance with one embodiment has a device body 14 with a rotatable clamp body 16 rotatably coupled to the body 14 at a rotatable joint 18 as shown. As best shown in FIGS. 1B and 2B, the device body 14 has a tube channel 20A defined within the body 14, while the clamp body 16 has a corresponding tube channel 20B defined within the clamp body 16. As such, when the clamp body 16 is in the clamped position as shown in FIGS. 1A and 2A, the two channels 20A, 20B form a tube lumen 20 through which a tube (such as tube 12) can be disposed and thereby clamped within the tube lumen 20. In addition, the device body 14 can also have a tube slot (or “opening”) 22 defined in the device body 14 and in communication with the tube channel 20A such that a tube (such as the tube 12 as shown in FIGS. 1A and 1B) can be urged into position within the tube channel 20A through the tube slot 22 (or, alternatively, such that device 10 can be urged over the tube 12 via the tube slot 22 such that the tube 12 is positioned within the tube channel 20A). Alternatively, the body 14 in certain implementations can have no tube slot.

One of skill in the art understands that the tube channels 20A, 20B can be channels, notches, indentations, or any other shapes defined within the device body 14 and the clamp body 16 such that the two shapes can form a lumen 20 or elongate opening shaped to receive a tube when the clamp body 16 is in the clamped position.

In one exemplary implementation as best shown in FIGS. 1B, 2B, and 3, the device body 14 has a base 30 that has two projections 32A, 32B that form the tube slot 22 discussed above. Further, the body 14 also has a top structure 34 and a middle structure 36 disposed between the base 30 and the top structure 34 as shown. Further, an outer wall 37 extends around an outer circumference of the body 14 and is attached to the base 30, the top structure 34, and the middle structure 36 as shown. Alternatively, the body 14 can be a substantially solid body 14 that has no separate components (no separate base 30, top structure 34, or middle structure 36). In a further alternative, the body 14 can be made up solely of a base 30 and a top structure 34.

In addition, the device body 14 can also have a pair of receiving slots 38A, 38B extending radially outward from the outer wall 37 of the device body 14 as shown. In the exemplary embodiment as shown, the receiving slots 38A, 38B are formed by two frames 40A, 40B extending from the device body 14. Alternatively, the slots 38A, 38B can be formed by or result from any known structures that can create such slots. Further, the outer wall 37 of the device body 14 has multiple attachment teeth 42 disposed on the outer wall 37 to which the clamp body prong 50 can be attached, as will be discussed in detail below. Alternatively, any known attachment mechanism or structure that can mateably attach to the prong 50 can be used.

According to certain embodiments as best shown in FIGS. 1B and 2B, the clamp body 16 has a prong (or protrusion) 50 extending from the clamp body 16 such that the prong 50 can be positioned through the two receiving slots 38A, 38B when the clamp body 16 is urged into the clamped position (as shown in FIGS. 1A, 2A, and 3). In addition, in some implementations, the prong 50 has teeth 52 disposed on an inner surface of the prong 50 such that the teeth 52 are mateable with the corresponding teeth 42 on the outer wall 37 of the device body 14. As such, when the clamp body 16 is urged toward the clamped position as shown in FIGS. 1A, 2A, and 3, the prong 50 is urged through at least the first receiving slot 38A such that the teeth 52 of the prong 50 are in contact with the teeth 42 of the outer wall 37.

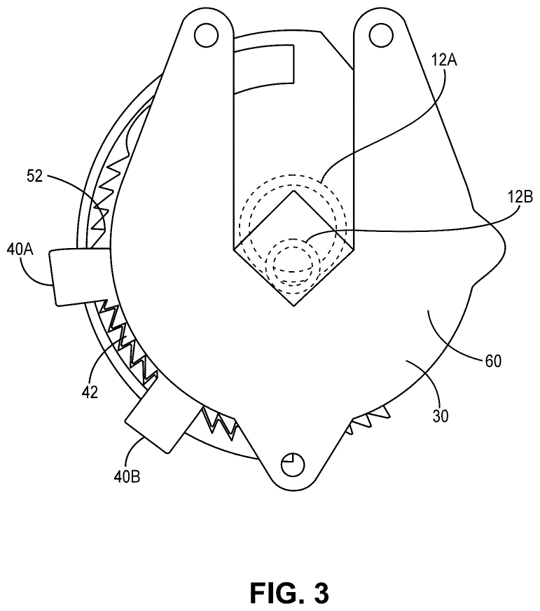

According to one embodiment, it is the prong 50 with the mateable teeth 52 that can couple with the teeth 42 of the outer wall 37 that results in the universality of the device 10. That is, the device 10 is adjustable to be used with tubes of multiple different sizes (diameters) as a result of the attachable prong 50. Once a tube (such as tube 12) is placed in the tube channel 20A of the device body 14, the clamp body 16 can be urged toward the clamped position. As the clamp body 16 moves toward the device body 14, the prong 50 is urged through the first receiving slot 38A and begins moving along the outer wall 37. Thus, as best shown in FIGS. 1A and 3, when the inner walls of the tube channel 20B of the clamp body 16 come into contact with the tube (such as tube 12), the clamp body 16 can be advanced until the frictional contact between the tube and the inner walls of both tube channels 20A, 20B is sufficient to fixedly attach the tube to the device 10. Thus, as shown in FIG. 3 with the two exemplary tube diameters 12A, 12B depicted, the clamp body 16 can be used to attach the device 10 to tubes with a wide variety of diameters. At this point, the teeth 52 of the prong 50 will be mateably attached to the teeth 42 of the outer wall 37 such that the prong 50 is attached to the outer wall 37 and thus the clamp body 16 is attached to the device body 14. Once the prong 50 is attached to the outer wall 37, the prong 50 can only be detached from the device body 14 by urging the prong 50 radially away from the outer wall 37 such that the teeth 52 detach from the teeth 42, thereby allowing the prong 50 and the clamp body 16 to be retracted toward the open position. Thus, in various implementations, the teeth 42, 52 (or other attachment structures as discussed below) are disposed along a length of the outer wall 37 and along a length of the prong 50, respectively, such that the prong 50 can attach to the device body 14 at various positions in order to accommodate tubes of a variety of diameters. In other words, for bigger tubes, the distal teeth 52 on the prong 50 will attach to teeth 42 on the wall 37, but for smaller tubes, the prong 50 will advance farther in relation to the device body 14 such that teeth 52 that are proximal of the distal teeth 52 will engage with teeth 42 on the wall 37.

In one embodiment, the mateable teeth 42, 52 are angled such that when the prong 50 is urged toward the clamped position, the angle of the two sets of teeth 42, 52 allows the prong 50 to be advanced while the teeth 42, 52 are in contact. However, the angle of the two sets of teeth 42, 52 are such that the prong 50 cannot easily be urged toward the open position. In other words, when the teeth 42, 52 are in contact, they prevent the prong 50 and thus the clamp body 16 from easily being urged toward the open position. Instead, as noted above, the entire prong 50 must be “lifted” or otherwise urged radially away from the device body 14 such that the teeth 42, 52 disengage from each other, thereby making it possible to urge the prong 50 and the clamp body 16 toward the open position.

In various alternative aspects, the attachment features or structures 42, 52 can be any known mechanisms, structures, or features 42, 52 for providing releasable attachment of the prong 50 to the device body 14 as described herein.

FIGS. 2A, 2B, and 3 depict an bottom view of the device 10, in which the bottom surface 60 of the base 30 is visible. In one embodiment, the bottom surface 60 of the base 30 has an attachment structure, mechanism, or feature (not shown) disposed thereon or incorporated therein. For example, in one specific implementation, the bottom surface 60 can have an adhesive on the surface 60 that can attach the base 30 to the patient's skin. In one specific exemplary embodiment, a medical-grade adhesive tape is disposed on the bottom surface 60 of the base 30. Alternatively, the bottom surface 60 can have microneedles or microhooks extending therefrom to provide for attachment to the skin. In a further alternative, the attachment component on the bottom surface 60 can be a combination of adhesive and microneedles or microhooks. In yet another exemplary alternative, any other known structure, mechanism, or feature for attachment to a patient's skin or body can be used.

In some implementations, the base 30 can also have three suture openings 62A, 62B, 62C defined within the base 30 as shown. Alternatively, the base 30 can have two, four, five, six, or any number of suture openings defined therein.

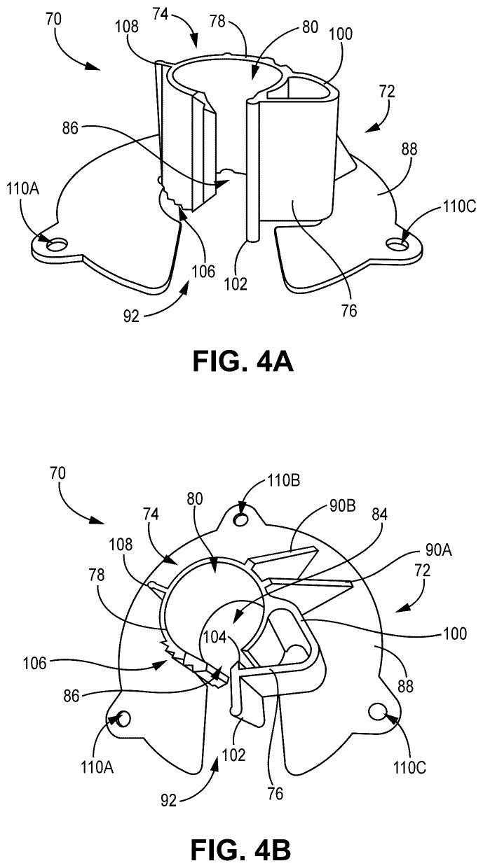

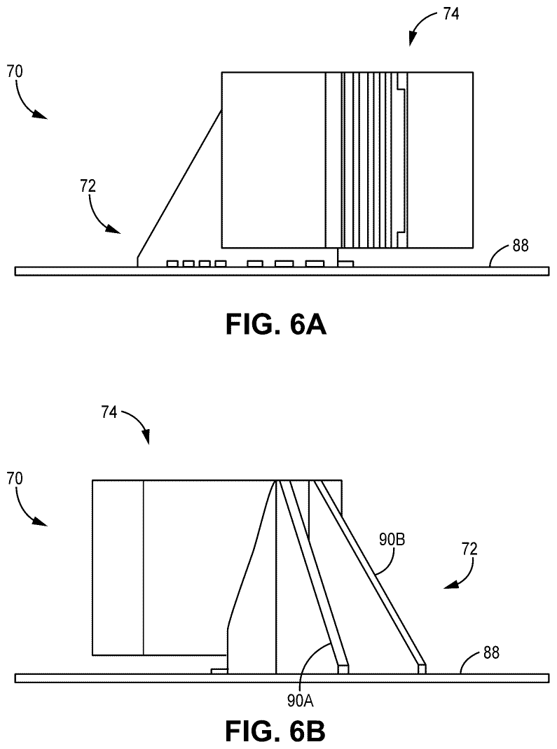

Another exemplary embodiment of a tube attachment device 70 is depicted in FIGS. 4A-6B. Except as expressly discussed below, the various components, features, methods of use, and other aspects of this implementation are the same or substantially similar to the device 10 discussed above. While FIGS. 4A-6B depict the device 70 in its open/unclamped position, one of ordinary skill understands that the device 70 also has a clamped or closed position (similar to device 10) as will be described in additional detail below, in accordance with another embodiment.

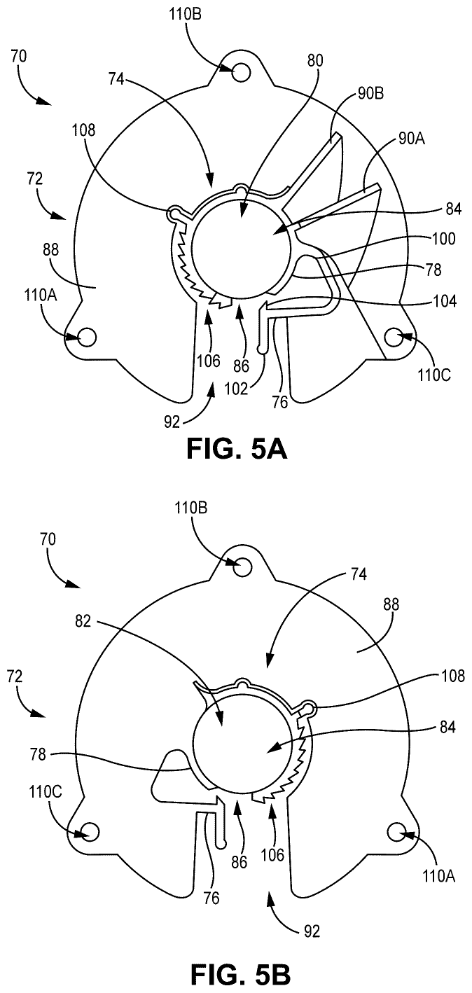

The device 70 in accordance with one embodiment has a device body 72 with an attachment ring 74 and an extendable clamp arm 76 coupled to the wall 78 of the ring 74 as shown. As best shown in FIGS. 4A-5B, the wall 78 of the attachment ring 74 defines a top opening 80 (as best shown in FIGS. 4A-4B), a bottom opening 82 (as best shown in FIG. 5B), and a lumen 84 (as best shown in FIGS. 4B, 5A, and 5B) extending from the top opening 80 to the bottom opening 82 such that the attachment ring 74 can receive a target tube that can be positioned therethrough. In addition, the ring 74 has an opening (or “ring slot”) 86 defined in the wall 78 that is configured to allow the ring 74 to be positioned around the target tube (not shown) as will be discussed in further detail below.

The device 70 also has a base 88 to which the attachment ring 74 is attached via attachment bodies 90A, 90B, as best shown in FIGS. 4B, 5A, and 6B. In this case, the exemplary attachment bodies 90A, 90B are triangular brackets 90A, 90B that couple the ring 74 to the base 88 and maintain the position of the ring 74 above the base 88 as shown. Alternatively, the ring 74 can be attached to the base 88 via a single attachment body, three bodies, or any number of attachment bodies, which can be brackets, other triangular-shaped structures, or any other known structure(s) for attaching the two components together.

In addition, the device body 72 can also have a tube slot (or “opening”) 92 defined in the base 88 and in communication with the ring lumen 84 such that a target tube can be urged into position within the lumen 84 of the ring 74 through the tube slot 92 (or, alternatively, such that device 70 can be urged over the tube via the tube slot 92 such that the tube is positioned within the lumen 84). Alternatively, the device 70 in certain implementations can have no tube slot.

Returning to the extendable clamp arm 76 as best shown in FIGS. 4A, 4B, and 5A, the arm 76 in this embodiment is a deformable structure 76 that is attached at its proximal end 100 to the wall 78 of the ring 74 and is disposed in a bent configuration in its resting or relaxed state as shown. Further, at its distal end, the arm 76 has an arm handle (or “grip”) 102 and an attachment projection 104 attached thereto, wherein the arm handle 102 extends from the arm 76 on the side facing generally away from the ring 74, while the projection 104 extends from the arm 76 on the side facing generally toward the ring 74. The arm handle 102 can be used to grasp the arm 76 when the arm 76 is being extended into its clamped position as discussed below, while the attachment projection 104 can be used to attach the arm 76 to the wall 78 of the ring 74 at a location on the opposite side of the opening 86 in the wall 78 (opposite of the side to which the arm 76 is attached). More specifically, in one embodiment, the outer surface of the wall 78 has an attachment structure 106 disposed thereon, to which the attachment projection 104 can be attached. In this specific exemplary implementation, the attachment structure 106 is made up of multiple attachment teeth 106 defined in or otherwise disposed along a length of the outer surface of the wall 78 to which the attachment projection 104 can be attached. Further, the ring wall 78 can also optionally have a wall handle 108 that can be grasped by the user when the clamp arm 76 and the wall 78 are being coupled together. As such, when a user grasps the arm handle 102 (and optionally the wall handle 108) and pulls the clamp arm 76 from its resting state across the opening 86 toward the attachment teeth 106, the projection 104 can be positioned to mateably attach to the teeth 106, thereby resulting in the clamp arm 76 being attached to the ring 74 such that the arm 76 extends across the opening 86, whereby the target tube disposed within the lumen 84 of the ring 74 is securely attached thereto.

In various alternative aspects, the attachment features or structures 104, 106 can be any known mechanisms, structures, or features 104, 106 for providing releasable attachment of the clamp arm 76 to the ring wall 78 as described herein.

The functionality of the extendable clamp arm 76 will now be described in additional detail. As noted above, the clamp arm 76 has a resting state as shown in the figures in which the clamp arm 76 is in its retracted or unclamped position (having a bent configuration as shown). Once the device 70 has been positioned around the target tube (not shown) such that the tube is disposed within the lumen 84 of the ring 74 as desired, the clamp arm 76 can be urged into its clamped position in the following fashion. The user can grasp the handle 102 and pull the clamp arm 76 out of its relaxed or resting state as it urges the distal end of the arm 76 across the opening 86 and adjacent to or into contact with the attachment teeth 106 on the ring wall 78. Because the arm 76 is being urged out of its resting state, the arm 76 begins to apply some resistive force against the pulling force being applied to extend the arm 76 to the attachment teeth 106, and the farther the arm 76 is extended or stretched, the greater the resistive force is generated by the arm 76. The increasing resistive or counter force generate by the arm 76 is a result of it being urged out of its bent configuration (into a straighter configuration) and as a result of it being deformed as the arm 76 is stretched out of its undeformed resting state. Thus, if the external force applied by the user were to be removed, the arm 76 would return to its resting, bent configuration as shown.

In certain embodiments, this resistive or return force being applied to urge the distal end of the arm 76 proximally can also facilitate its attachment to the attachment teeth 106. That is, as with the mateable teeth 42, 52 discussed above, the attachment teeth 106 and the attachment projection 104 can be angled such that when the extendable arm 76 is urged toward the clamped position, the angle of the teeth 106 and the projection 104 allows the arm 76 to be advanced while the attachment structures 106, 104 are in contact. However, once the projection 104 is attached to the teeth 106, the angle of the structures 106, 104 are such that the arm 76 cannot easily be urged toward the open position. In other words, when the projection 104 and teeth 106 are coupled, they prevent the clamp arm 76 from easily being urged toward the open position. Instead, in a similar fashion to the other device embodiment (device 10), the entire arm 76 must be “lifted” or otherwise urged radially away from the ring wall 78 such that the projection 104 disengages from the teeth 106, thereby making it possible to allow the extendable arm 76 to return to its open or retracted position as a result of the tensioned forces that urge it toward its relaxed or resting state.

According to one embodiment, as with the device 10 discussed above, it is the coupling mechanism of the extendable arm 76 being coupleable via the projection 104 to the teeth 106 of the ring wall 78 that results in the universality of the device 70. That is, the device 70 (like device 10) is adjustable to be used with tubes of multiple different sizes (diameters) as a result of the deformable ring wall 78 and the extendable arm 76. Once a tube (such as tube 12 as discussed above with respect to device 10) is placed in the lumen 84 of the ring 74, the clamp arm 76 can be urged toward the clamped position. As the user grasps the arm handle 102 (and optionally the ring handle 108), the user urges clamp arm 76 toward the attachment teeth 106 of the ring wall 78 and thereby urges the opposing portions of the wall 78 (on opposing sides of the opening 86) together such that lumen 84 is reduced in size, thereby urging the inner surfaces of the wall 78 into contact with the tube (such as tube 12) disposed therein. Thus, as the inner surfaces of the wall 78 come into contact with the tube (such as tube 12), the clamp arm 76 is advanced distally until the frictional contact between the tube and the inner surfaces of the wall 78 is sufficient to fixedly attach the tube to the device 70. Thus, the ring 74 and the extendable arm 76 can be used to attach the device 70 to tubes with a wide variety of diameters. At this point, the projection 104 of the arm 76 will be mateably attached to the teeth 106 of the wall 78 such that the arm 76 is attached to the wall 78. Thus, in various implementations, the teeth 106 (or other attachment structures) are disposed along a length of the wall 78 such that the arm 76 can attach to the wall 78 at various positions in order to accommodate tubes of a variety of diameters. In other words, for bigger tubes, the projection 104 will attach to teeth 106 near the opening 86, but for smaller tubes, the projection 104 will be urged farther in relation to the ring 74 such that the projection 104 will engage with teeth 106 farther from the opening 86 on the wall 78, which also causes the lumen 84 to have a smaller diameter (and thus be in sufficient contact with the smaller tube) as discussed above.

As with the device 10, the underside of the base 88 (as best shown in FIG. 5B) has an attachment structure, mechanism, or feature (not shown) disposed thereon or incorporated therein, in the same fashion as the device 10. Further, the base 88 in this embodiment can also have three suture openings 110A, 110B, 110C defined within the base 88 as shown which can be used to attach the device 70 to the patient via sutures, staples or the like. Alternatively, the base 88 can have two, four, five, six, or any number of suture openings defined therein.

In accordance with certain embodiments, the various components of the device implementations herein (including, for example, device 10 and device 70), can be made of semi-flexible polypropylene. Alternatively, any device herein can be made of any appropriate polymeric material (such as nylon, other synthetic polymers, or the like) or any appropriate metal or any other appropriate material or combination of materials for use in medical devices. Further, in some aspects, the diameter of the base 30 can range from about 2 inches to about 5 inches. Alternatively, the base 30 can have a diameter of about 3.5 inches. In addition, the device 10 can have a height ranging from about 1 inches to about 2 inches. Alternatively, the height of the device 10 can be about 1.5 inches. In yet another alternative embodiment, the device 10 can have any dimensions that allow for use in various different applications as described herein depending on the dimensions of the tube to which the device 10 will be attached. For example, the various device implementations disclosed or contemplated herein can be attachable to any tube ranging in size from about 16 French to about 36 French. Alternatively, the target tube can range in size from about 20 Fr to about 24 Fr, from about 28 Fr to about 36 Fr, or from about 32 Fr to about 36 Fr. Alternatively, any device embodiment herein can be sized to be used with any tube of any known size.

In use, once the tube is inserted into the patient's chest or other body part as desired, the device 10, 70 is placed in the open configuration (as shown in FIGS. 1B and 2B for device 10 and in FIGS. 4A-5B for device 70) and the device 10, 70 is urged toward the tube (such as tube 12) with the tube positioned within the tube slot 22, 92 such that the tube ultimately is disposed within the tube channel 20A of device 10 or the lumen 84 of device 70. Further, the base 30, 88 is also positioned adjacent to or in contact with the skin of the patient in proximity to the tube 12. At this point, the device 10, 70 is urged into its clamped configuration. More specifically, in device 10, the clamp body 16 is urged toward the clamped position until both channels 20A, 20B are in sufficient frictional contact with the tube, and the prong 50 is attached to the outer wall 37 via the teeth 42, 52 or other attachment structures or features. And with device 70, the clamp arm 76 is urged toward the teeth 106 (and the teeth 106 can be urged toward the clamp arm 76 via the optional ring handle 108 as well) until the inner surfaces of the ring wall 78 are in sufficient frictional contact with the tube, and the projection 104 is attached to the teeth 106 or other attachment structures or features. The device 10, 70 can then be attached to the skin of the patient via any of the structures, mechanisms, or features discussed above or any other known options. Once the device 10, 70 is attached to the tube 12 and the skin of the patient, the device 10, 70 can serve to stabilize and/or maintain the position of the tube 12 in relation to the patient. In other words, the device 10, 70 can keep the tube attached to the patient for the desired length of time. When it is time to remove the tube from the patient, the device 10, 70 can be detached from the tube 12 and the patient. That is, in device 10, the prong 50 can be urged radially away from the device body 14 and thereby is released from its attachment thereto such that the clamp body 16 can be urged toward and into its open position, thereby releasing the tube 12 from the device 10. Similarly, in device 70, the clamp arm 76 is urged radially away from the ring wall 78 and thereby is released from its attachment thereto such that the clamp arm 76 is urged by its internal tension toward its resting state in its open or unclamped position, thereby releasing the tube 12 from the device 70. At this point, the device 10, 70 can also be detached from the skin of the patient, such as by applying sufficient force to overcome the strength of the adhesive or other attachment component and thereby detaching the device from the patient's skin.

In addition to use with chest tubes, the various device embodiments herein can also be used with dialysis tubes, peripheral inserted central catheter lines, cranial procedures, and any other applications or procedures, including drainage of fluids from other, non-chest locations, in which the insertion of an externally connected tube or tubular device is required.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A tube attachment device comprising:
(a) a tube attachment ring, the ring comprising:
(i) a deformable wall;
(ii) a lumen defined through the deformable wall;
(iii) a side opening defined in the deformable wall, wherein the side opening is in fluidic communication with the lumen; and
(iv) a wall attachment structure disposed on an outer surface of the deformable wall;
(b) an extendable clamp arm operably coupled to the tube attachment ring, the extendable clamp arm comprising an arm attachment structure disposed on the extendable clamp arm, wherein the arm attachment structure is coupleable with the wall attachment structure, wherein the extendable clamp arm is moveable between a retracted position and an extended position in which the arm attachment structure is coupled to the wall attachment structure and the extendable clamp arm is disposed across the side opening; and
(c) a base operably coupled to the tube attachment ring, the base comprising a device body attachment component on an underside of the base.

2. The tube attachment device of claim 1, wherein the wall attachment structure comprises a plurality of teeth.

3. The tube attachment device of claim 2, wherein the arm attachment structure comprises a projection, wherein the projection is coupleable with the plurality of teeth.

4. The tube attachment device of claim 1, wherein the extendable clamp arm comprises a handle disposed on the extendable clamp arm.

5. The tube attachment device of claim 1, wherein the extendable clamp arm in the retracted position is in an untensioned state, and wherein the extendable clamp arm in the extended position is in a tensioned state.

6. The tube attachment device of claim 1, wherein the device body attachment component is configured to be attachable to a patient's skin.

7. A tube attachment device comprising:
(a) a device body comprising:
(i) a first channel defined within the device body;
(ii) a device base comprising a device body attachment component on an underside of the device base; and
(iii) a first attachment structure disposed on the device body;
(b) a clamp body rotatably coupled to the device body via a rotatable joint, wherein the clamp body is rotatable between an open position and a clamped position, the clamp body comprising:
(i) a second channel defined within the clamp body, wherein the second notch is parallel with the first channel; and
(ii) a prong extending from the clamp body, wherein the prong comprises a second attachment structure, wherein the second attachment structure is removably coupleable with the first attachment structure,
wherein, when the clamp body is in the clamped position, the first and second channels form a tube lumen sized and shaped to receive a target medical tube such that the device body and the clamp body frictionally attach the tube attachment device to the target medical tube.

8. The tube attachment device of claim 7, wherein the device body further comprises a tube slot defined within the device base, wherein the tube slot is in communication with the first channel, wherein the tube slot has a length that is transverse to the longitudinal axis of the first channel.

9. The tube attachment device of claim 7, wherein the device body further comprises an outer wall disposed on the device base, wherein the first attachment structure is disposed on the outer wall.

10. The tube attachment device of claim 7, wherein the first attachment structure and the second attachment structure comprise mateable teeth.

11. The tube attachment device of claim 7, wherein the prong is curved to substantially match a curvature of an outer wall of the device body.

12. The tube attachment device of claim 7, wherein the device body further comprises at least one prong receiving slot associated with the device body, wherein the at least one prong receiving slot is configured to receive the prong as the clamp body is moved into the clamped position.

13. The tube attachment device of claim 7, wherein the device body attachment component is configured to be attachable to a patient's skin.

14. The tube attachment device of claim 7, wherein the device body attachment component comprises at least one of an adhesive, microneedles, and microhooks.

15. A method of ensuring a medical tube inserted into a patient remains attached to the patient, the method comprising:
urging a tube attachment device toward the medical tube extending from the patient, wherein the tube attachment device comprises:
(a) a tube attachment ring comprising:
(i) a lumen defined within the tube attachment ring;
(ii) a side opening defined in the side of the tube attachment ring, wherein the side opening is in fluidic communication with the lumen; and
(iii) a first attachment structure disposed on the tube attachment ring; and
(b) an extendable clamp arm operably coupled to the tube attachment ring, wherein the extendable clamp arm is movable between an open position and a clamped position, the extendable clamp arm comprising:
(i) a second attachment structure disposed on the extendable clamp arm;
positioning the tube attachment ring such that the medical tube is disposed within the lumen through the side opening;
urging the extendable clamp arm toward the clamped position until the tube attachment ring is in sufficient frictional attachment with the medical tube such that the tube attachment device is attached to the medical tube;
attaching the extendable clamp arm to the tube attachment ring in the clamped position by attaching the first attachment structure and the second attachment structure; and
attaching the tube attachment device to the patient.

16. The method of claim 15, wherein the urging the extendable clamp arm toward the clamped position further comprises urging the extendable clamp arm into a position in relation to the tube attachment ring such that the second attachment structure is coupled with the first attachment structure, whereby the extendable clamp arm is removably attached to the tube attachment ring.

17. The method of claim 15, wherein the tube attachment device further comprises a base operably coupled to the tube attachment ring, wherein the base comprises a tube slot in communication with the lumen, wherein the positioning the tube attachment ring such that the medical tube is disposed within the lumen through the side opening further comprises positioning the base such that the medical tube is urged into the tube slot and toward the side opening and the lumen.

18. The method of claim 15, wherein the tube attachment device further comprises a base operably coupled to the tube attachment ring, wherein the attaching the tube attachment device to the patient further comprises attaching an underside of the base to skin of the patient via an adhesive.

19. The method of claim 15, wherein the medical tube comprises a chest tube, a dialysis tube, a catheter line, or a tube for a cranial procedure.

20. The method of claim 15, further comprising detaching the extendable clamp arm from the tube attachment ring by urging a distal end of the extendable clamp arm radially away from the tube attachment ring, whereby the second attachment structure disengages from the first attachment structure.
